(19) United States
(12) Patent Application Publication
(10) Pub. No.: US 2013/0239331 A1
(43) Pub. Date: Sep. 19, 2013

(54) Inflation Support System for MR Guided HIFU

(71) Applicants: John W. EATON, Palo Alto, CA (US); Keith A. WONG, San Francisco, CA (US)

(72) Inventors: John W. EATON, Palo Alto, CA (US); Keith A. WONG, San Francisco, CA (US)

(73) Assignee: Siemens Medical Solutions USA, Inc., Malvern, PA (US)

(21) Appl. No.: 13/424,123

(22) Filed: Mar. 19, 2012

(51) Int. Cl. A61B 6/04; A61G 13/02

(52) U.S. Cl. 5/601

(57) ABSTRACT

A support device has an inflatable element positioned above a top side of a table, above which a patient can be positioned. The table is part of, and movable into and out of a bore of, a medical imaging machine. The inflatable element can have one or more fluid fillable cells selectively inflatable and deflatable to alter a position of a patent relative to the top side of the table. The support device can be employed in a system having a control apparatus coupled to the support device and operable to inflate and deflate the inflatable element to alter the position of a patient supported by the table.

### BACKGROUND

The disclosed embodiments relate to patient positioning within a medical imaging machine used during medical procedures.

A patient is typically placed on a table and then the table is moved inside a bore of a medical imaging machine, such as a magnetic resonance (MR) machine, when undergoing a medical procedure within the machine. The patient, and particularly the targeted tissue, organ, or body part should be positioned accurately within the machine to achieve satisfactory results. This is particularly true for MR guided, high-intensity focused ultrasound (HIFU) procedures.

MR guided HIFU is a highly precise medical procedure using high-intensity focused ultrasound energy. During this type of therapeutic ultrasound procedure, the acoustic energy from the HIFU transducer is guided by MR imaging and can rapidly heat and destroy diseased tissue via ablation. Clinical HIFU procedures are typically image-guided to permit treatment planning and targeting of specific tissue or organs before a therapeutic or ablative level of ultrasound energy is applied.

Therapeutic ultrasound is a minimally invasive or non-invasive method to deposit acoustic energy into tissue. MR guided HIFU requires that the targeted tissue of the patient be positioned very accurately relative to the HIFU transducer while the patient is in either a supine or prone position. In some situations, the patient may also be oriented obliquely (along the cranio-caudal axis) to allow the ultrasound energy better access to organs or the tissue to be treated. The HIFU transducer must be coupled to the patient to ensure efficient transmission of the acoustic energy. There are many ways to achieve this including use of an acoustic pad, a water bolus (contained within a thin membrane), or direct skin contact with the transducer. Ultrasound gel aids in the acoustic coupling. The acoustic beam must also be directed or steered (mechanically or electronically) accurately at the tissue to be treated.

Patients vary greatly in size, shape, and weight. Each patient must be carefully positioned and adjusted on the table before entering the MR bore of the machine. Typically, once inside the bore, the patient cannot be repositioned, at least not accurately. Thus, if the patient is poorly positioned, the results of the procedure will not meet expectations. Alternatively, the patient can be extracted from the bore, repositioned, and then moved back into the bore. Existing tables have been modified to include wood or plastic fixtures covered in plastic to assist in patient positioning. Such fixtures must be non-magnetic and MR compatible. Such fixtures typically require a highly iterative process to get the patient positioned correctly. The patient typically must get up from and lie down on the table multiple times or, if sedated, be moved by a nurse or technician as the fixture is adjusted. This can be time consuming, resulting in delays in procedures, increased expense, and the like. Also, the design of such fixtures and the time consuming nature of the trial and error adjustment process do not allow for fine tuning the patient position to achieve an optimum position for a given procedure.

### BRIEF SUMMARY

The preferred embodiments described below include methods, systems, and devices for positioning a patient within a medical imaging machine, such as a MR machine. Such machines are used to perform therapeutic procedures on a patient as well as to acquire images of specific anatomical aspects of a patient, and/or the like. The position of the patient relative to the energy source can be important. This is particularly true in a MR guided HIFU machine set up, where patient positioning relative to the HIFU transducer is highly important in order to obtain or provide the best therapeutic results.

In one embodiment, a system is disclosed for adjusting a position of a patient and a HIFU transducer relative to one another in a medical imaging machine having a table. The system has an adjustable support device including an inflatable element. The system also has a control apparatus coupled to the adjustable support device and operable to inflate and deflate the inflatable element to alter the position of the patient supported by the table and the HIFU transducer relative to one another.

The adjustable support device can be arranged to move the patient or to move the HIFU transducer.

When arranged to move the patient, the inflatable element can include one or more fluid fillable cells arranged to raise or lower the patient relative to the table. The one or more inflatable cells can be arranged to adjust the orientation of the patient about a lengthwise axis of the table.

The inflatable element can include at least one fluid fillable cell arranged to move a patient laterally relative to the table. The inflatable element can include at least one fluid fillable cell arranged to move a patient lengthwise along an axis of the table.

The adjustable support device can include a carrier positioned beneath the inflatable element and portions of the inflatable element can be secured to the carrier. The carrier can have a horizontal panel and a vertical panel along at least one edge of the horizontal panel. The inflatable element can have a plurality of fluid fillable cells that can be connected or tethered to the carrier resulting in movement of the plurality of cells relative to portions of the carrier when the cells are inflated and deflated.

When arranged to move the HIFU transducer, the HIFU transducer can be coupled to the adjustable support device. The adjustable support device can also be mounted to a surface within a bore of the medical imaging machine.

The control apparatus can include a pump or compressor; one or more valves; and one or more lines each in fluid communication with part of the inflatable element, the pump or compressor, and a corresponding valve of the one or more valves. The pump or compressor and valves can be operable to inflate and deflate the inflatable element of the adjustable support device.

The inflatable element can have one or more vertically adjacent layers of cells and/or can have one or more horizontally adjacent layers of cells.

The adjustable support device can have a length and the inflatable element can have one or more fluid fillable cells each having an interior fluid space extending substantially the length of the adjustable support device.

The medical imaging machine may be an MR machine.

In another embodiment, a support device for a medical imaging machine has a bore and a table movable into and out of the bore. The table has a top side above which the patient is positioned. The support device has an inflatable element positioned above the top side of the table and has a plurality of fluid fillable cells that are selectively inflatable and deflatable to alter a position of a patent relative to the top side of the table.

The support device can have a patient platform carried on top of the plurality of fluid fillable cells. The patient platform can be a wedge-shaped structure having a top surface oriented at a side-to-side angle relative to a horizontal reference and the top side of the table. The patient platform can be a flat, contoured, wedge, or otherwise shaped structure with a top surface oriented parallel or at a side-to-side angle relative to a horizontal reference and to the top side of the table.

The support device can include at least one fluid fillable cell positioned along a side of the table arranged to laterally move or position a patient side to side when inflated or deflated. The support device can include at least one fluid fillable cell positioned along a lengthwise end of the table arranged to move or position a patient in a lengthwise direction when inflated or deflated.

A substantial portion of the plurality of fluid fillable cells can be positioned and arranged to raise and lower a patient relative to the top side of the table and/or to alter the angle of a patient about a lengthwise axis and/or a width-wise axis of the support device when inflated and deflated.

The support device can include a carrier mounted to the table and the inflatable element can be connected to the carrier. A portion of the plurality of fluid fillable cells can be secured to the table or to the carrier to maintain a position of the cells relative to the table.

In another embodiment, a method is provided for positioning a patient on a table movable into and out of a bore of a medical imaging machine. The method includes mounting an adjustable support device above a top side of the table, the adjustable support device having one or more fluid fillable cells. A patient is then positioned on the adjustable support device. A control apparatus is operated, either manually, partially automatically, or automatically, to selectively inflate, deflate, or both, any one or more of the one or more of the fluid fillable cells to adjust the position of the patient relative to the top side of the table.

In another embodiment, a method is provided for positioning a HIFU transducer and a patient relative to one another within a bore of a magnetic resonance machine. The method includes positioning a patient on a table; moving the table into the bore, and operating a control apparatus to selectively inflate, deflate, or both, any one or more of a plurality of fluid fillable cells to adjust the position of the patient and HIDU transducer relative to one another. The step of operating the control apparatus can include moving the patient relative to the HIFU transducer. Alternatively, the step of operating the control apparatus can include moving the HIFU transducer relative to the patient.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a schematic, generic representation of a MR machine with a patient table and an adjustable support device constructed in accordance with the teachings of the present invention.

FIG. 2 is a cross-section of the patient table and bore of the machine shown in FIG. 1 with the table and patient disposed within the bore and with all of the inflatable cells of the adjustable support device inflated.

FIG. 3 is a perspective view of the MR machine and table represented in FIGS. 1 and 2.

FIG. 4 is a cross-section of the table and bore shown in FIG. 2, but with a portion of the inflatable cells of the adjustable support device deflated.

FIG. 5 is a cross-section of the table and bore shown in FIG. 4, but with an additional portion of the inflatable cells of the adjustable support device partially deflated.

FIG. 6 is a cross-section of the table and bore shown in FIG. 2, but with another portion of the inflatable cells of the adjustable support device deflated.

FIG. 7 is a cross-section of the table and bore shown in FIG. 2, but with another portion of the inflatable cells of the adjustable support device partially deflated.

FIG. 8 is a cross-section of the table and bore shown in FIG. 6, but with another portion of the inflatable cells of the adjustable support device deflated.

FIG. 9 is a cross-section of the table and bore shown in FIG. 8, but with yet another portion of the inflatable cells of the adjustable support device deflated.

FIG. 10 is a cross-section of a MR machine bore with another example of a patient table and adjustable support device in accordance with the teachings of the present invention.

FIG. 11 is a cross-section of a MR machine bore with another example of a patient table and adjustable support device in accordance with the teachings of the present invention.

FIG. 12 is a cross-section of a MR machine bore with another example of a patient table and adjustable support device in accordance with the teachings of the present invention

FIG. 13 is a cross-section of a MR machine bore with another example of a patient table and adjustable support device in accordance with the teachings of the present invention

FIG. 14 is a cross-section of a MR machine bore with another example of a patient table and adjustable support device in accordance with the teachings of the present invention

FIG. 15 is a plan view of another example of an adjustable support device for a patient table in accordance with the teachings of the present invention.

FIG. 16 is a lengthwise cross-section along another example of an adjustable support device in accordance with the teachings of the present invention.

FIG. 17 is a cross-section of a MR machine bore with another example of an adjustable support device in accordance with the teachings of the present invention.

### DETAILED DESCRIPTION OF THE DRAWINGS AND PRESENTLY PREFERRED EMBODIMENTS

MR guided HIFU procedures are used to locate diseased tissue and to direct high-intensity ultrasound energy at the tissue. Precise and repeatable patient positioning is important to obtain accurate and desired outcomes or results during such procedures. Known patient tissue positioning procedures and devices are inadequate to enable efficient and repeatable positioning from the initial diagnostic scans to the subsequent treatment scans. No procedures or devices are known that allow for reposition a patient or patient tissue once inside the bore of a MR machine.

Inflatable support devices, methods, and systems capable of repositioning a patient on a patient table are disclosed herein. The inflatable support may be particularly useful during MR guided HIFU procedures in a MR machine. The inflatable support permits repositioning the patient or the HIFU transducer while the patient is on the table, and whether the patient and table are within the bore or outside the bore. The inflatable support can produce elevation change, rotational or angular reorientation, lateral position adjustment, and/or lengthwise or longitudinal position adjustment.

A typical MR machine generally has a cryomagnet, gradient coil, and body coil in an RF cabin, such as a room isolated by a Faraday cage. A tubular or laterally open examination subject bore encloses a field of view. A more open arrangement may be provided. A patient bed (e.g., a patient gurney or table) supports an examination subject, such as a patient with or without one or more local coils. The patient bed may be moved into the examination subject bore in order to generate images of the patient. Received signals may be transmitted by the local coil arrangement to the MR receiver via, for example, coaxial cable or radio link (e.g., via antennas) for localization.

Other parts of a typical MR machine can be provided within a same housing, within a same room (e.g., within the radio frequency cabin), within a same facility, or connected remotely. The other parts of the MR machine may include local coils, cooling systems, pulse generation systems, image processing systems, and user interface systems. Any now known or later developed MR imaging system may be used. The location of the different components of the MR machine is within or outside the RF cabin, such as the image processing, tomography, power generation, and user interface components being outside the RF cabin. Power cables, cooling lines, and communication cables connect the pulse generation, magnet control, and detection systems within the RF cabin with the components outside the RF cabin through a filter plate. The MR machine can be configured by software, hardware, or both to acquire data representing a plane or volume in the patient. In order to examine the patient, different magnetic fields are temporally and spatially coordinated with one another for application to the patient.

The MR machine may be configured to acquire different types of data. For example, the MR data may represent the anatomy of the patient. The MR data may represent the response to the magnetic fields and radio-frequency pulses of tissue. Any tissue may be represented, such as soft tissue, bone, or blood. The disclosed methods, systems, and devices are suitable for use for many different types of MR machine use.

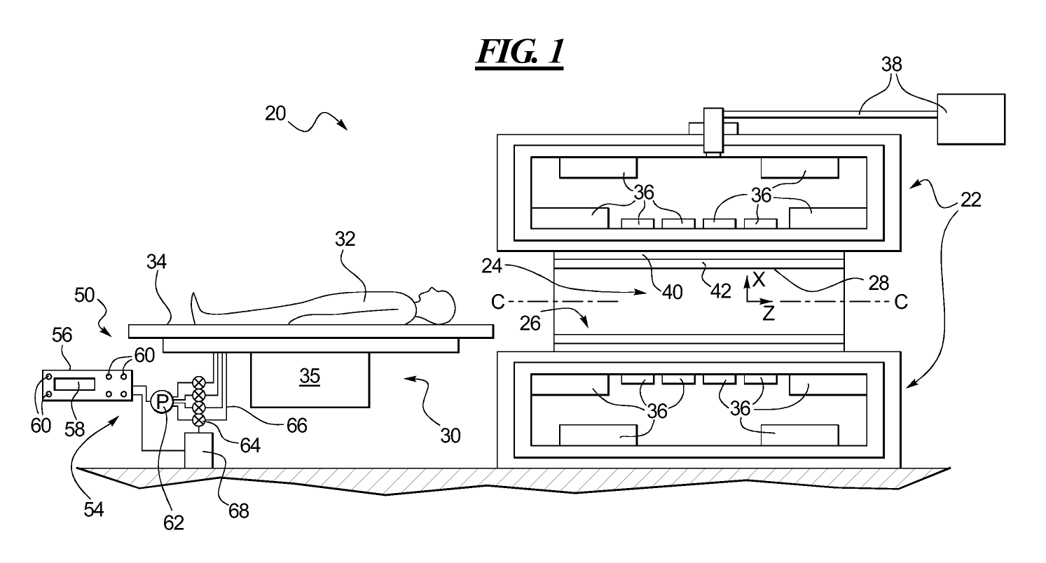

FIG. 1 shows a generic representation of a MR machine 20. The MR machine 20 generally has a base body 22 that defines an examination region 24 within a bore 26, as is known in the art. The examination region 24 and the bore 26 are typically formed symmetrically around a central axis C defined therein. The bore 26 is open axially at both ends relative to the central axis C along the direction of the axis. The bore 26 has an interior wall 28 extending circumferentially around and radially spaced from the central axis C.

The interior wall 28 of the bore 26 is typically a specified distance from the central axis C. This distance can be a constant such that the bore is, in fact, circular in cross-section. The diameter of the bore in this type of MR machine is typically about 50 cm to about 70 cm. However, the bore can have an elliptical or oval shape in cross-section in some instances. The interior wall 28 is typically closed around the central axis C.

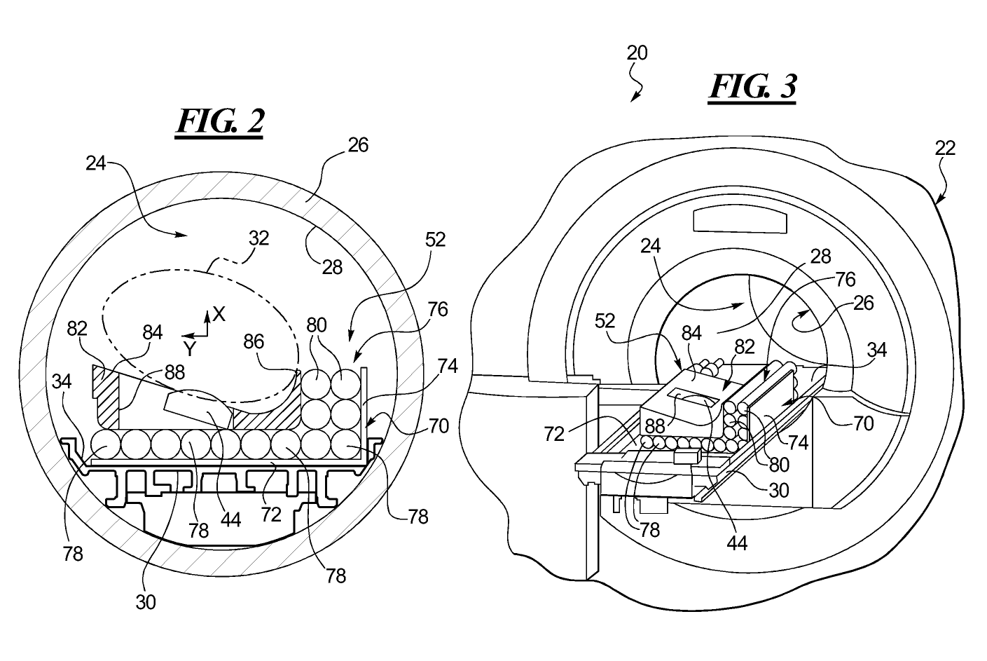

The MR machine 20 also generally has a transport bed or table 30. The table 30, together with a patient 32 lying above a top side 34 of the table, can be conveyed into the examination region 24 in the bore 26, as depicted in FIG. 2. The table 30 can be conveyed by a motor 35 or other MR compatible suitable drive mechanism. The typical MR machine 20 also has a basic field magnet 36, such as the aforementioned cryomagnet, associated with the bore 26, as is known in the art. In one example, the basic field magnet 36 can have a system of ring magnets 38 arranged concentric with the central axis C of the machine 20. Elliptical or oval ring magnets are also possible. Various arrangements with this type of MR machine 20 can produce a static magnetic field that is essentially homogeneous within the examination region 24 and generated by the basic field magnet 36. The magnetic field typically runs parallel to the central axis C. However, other embodiments are possible wherein the basic magnetic field is perpendicular to the central axis.

The basic field magnet 36 can be a permanent magnet, an electromagnetic, a cryomagnet, a superconducting magnet, or the like. A cooling device 38 can be associated with the basic field magnet 36 to circulate a cooling medium, such as liquid air or liquid nitrogen, to cool components of the MR machine 20 including the basic field magnet 36. A transmission arrangement 40 and a reception arrangement 42 are positioned radially outward, abutting the interior wall 28 of the bore and surround the outside of the examination region 24.

The foregoing described generic elements of the MR machine 20 can vary within the spirit and scope of the present invention. These elements are not particularly essential to the inventive aspects of the disclosed methods, systems, and devices for positioning a patient. The configuration and construction of the MR machine 20 can vary from the embodiment shown and described herein.

As noted above, the MR machine 20 can be used for imaging procedures or for therapeutic procedures. As shown in FIGS. 2 and 3, the machine can be equipped with a HIFU transducer 44, inclusive or one or more ultrasound transducers, to treat surface tissue, depth tissue, organs, and the like. The HIFU transducer 44 can be guided by the MR imaging function of the MR machine 20, as is known in the art. For optimum performance, the MR guided HIFU transducer 44, acoustic window of the transducer, pad, water bolus, or any combination thereof should contact the patient's skin and be positioned or aligned as close as possible to and in direct line with the tissue to be thermally ablated or treated. The disclosed systems, devices, and methods are configured to allow repositioning of a patient lying on the table 30 while the table is positioned in the examination region 24 of the bore 26.

In that regard, a patient positioning system 50 has a support device 52 and each is constructed in accordance with the teachings of the present invention. In particular, FIGS. 1-3 generally show the components of the disclosed system 50 and support device 52. The system 50 includes a control apparatus 54 coupled to and in fluid communication with the support device 52. The components and specifics of the control apparatus 54 can vary from the example shown and described herein. In addition, the system can utilize air or another suitable gas or liquid to operate the support device 52.

In one example, the control apparatus 54 has a user interface 56 with a display or graphical interface 58 and a number of controls 60. The control apparatus 54 also has one or more pumps or compressors 62 (though only one is shown and mentioned) and a series of control valves 64. A plurality of fluid lines 66 are connected to the pump or compressor 62 and each line is connected to a corresponding one of the series of valves 64. The control valves 64 can be operated by a controller 68 to open and close the fluid lines during use. The fluid lines 66 are also connected to the support device 52 as described below. These control apparatus components will typically be situated well outside the magnetic field of the MR machine or outside of the faraday cage of the MR suite.

The control apparatus 54 can take on any number of configurations and constructions and yet function as intended. In general, the control apparatus 54 is intended to permit a user to operate and control, i.e., inflate and deflate, portions of the support device 52 for positioning a patient on the table 30 or positioning the patient and HIFU transducer relative to one another. The user can manipulate the controls 60 on the user interface 56 to control the patient positioning system. The user can view performance information and data on the display 58 to monitor various system performance characteristics and operational parameters. The controller 68 can be configured to be completely manual and operable by a technician through the user interface 56. More likely, the controller can be partially or fully automated, be operable via a computer or processor, and be programmed to automatically control the pump or compressor 62 and the series of valves 64. These control apparatus components are operable to control the support device 52 through the fluid lines 66 to adjust the patient position on the table 30 in this example.

FIGS. 2 and 3 show that the table 30 can be a conventional MR machine table or transport bed. The support device 52 can be mounted to or carried on the table 30. In this example the support device 52 has a carrier 70 lying on or mounted to the top side 34 of the table 30. The carrier 70 has a horizontal panel 72 resting directly on the table 30 and has a vertical panel 74 extending upward along one side edge of the horizontal panel. Though only one vertical panel is shown herein, the carrier may include such a vertical panel on each side of the horizontal panel to achieve full positional control of the support device. The support device 52 also has an inflatable element, such as an inflatable bed 76 disposed on and/or connected to the carrier 70. In this example, the inflatable bed 76 has a plurality of inflatable or fluid fillable cells, bladders, or chambers (hereinafter referred to primarily as cells). A single row of the cells 78 lies across and against the horizontal panel 72 and two adjacent columns of the cells 80 are positioned adjacent the vertical panel 74 of the carrier.

In this example, the support device 52 also has an optional patient platform 82. The platform 82 can be formed of a dense foam material and have a side-to-side angled top surface 84. The angled top surface 84 can configure the platform having a wedge shape when viewed on end. Certain procedures may require that the patient 32 be positioned at an angle or rotated about the center axis C. The platform 82 can be utilized to macro-position the patient at such an angle. In this example, the platform 82 is positioned on top of the row of cells 78 and has one side wall 86 against one of the two columns of cells 80. The side wall 86 can assist in retaining a patient resting on the platform 82 is stationary position on the angled top surface 84. In this example, the platform 82 also has a central opening 88 in the angled top surface 84 and through the depth of the body of the platform. The HIFU transducer 44 is positioned in the central opening 88 in this example.

The HIFU transducer 44 can be composed of individual transducers or an array of transducers so that the therapeutic beams can be steered precisely and directed to the selected treatment area or tissue of the patient. The transducer applicator is typically held in place by a separate support structure which rests on the table (not shown herein). Alternatively, this support structure can also be integrated into the carrier 70, if desired. This can allow for consistent and repeatable positioning of the applicator relative to the patient and can also make the applicator more secure with respect to the carrier. In addition to the adjustment of the patient position by the fluid fillable cells, this support structure can be made adjustable in height toward the patient or tilted side to side or in the lengthwise axis of the table, thereby working in conjunction with the disclosed inflation systems, devices, and methods to provide the ability to insure proper positioning for almost any patient size.

Each fluid line 66 of the plurality of fluid lines is coupled to and in fluid communication with a corresponding one of the fluid fillable cells 78, 80. The cells 78, 80 can be inflated and deflated as needed by introducing air into the cells and evacuating air from the cells. The controller 68 can be operable to control the series of valves 64 independent of one another. As a result, each of the cells 78, 80 can be inflated and deflated independent of the other cells.

All of the components of the control apparatus 54, except for the fluid lines 66, can be positioned well outside of the examination region 24 and the bore 26. In one example, these components can be positioned in a separate control room and the bore can be in an MR suite adjacent the control room. The fluid lines 66 can be formed of plastic or other MR suitable material. Likewise, the inflatable bed 76, carrier 70, and platform 82 can be formed of non-metallic materials compatible for use within the MR examination region 24. The fluid lines 66 should be sufficiently lengthy and routed so as to allow the table 30 to move through the examination region 24 and bore 26 without interfering with the operation of the base magnetic field.

The adjustable support device 52 can be operated by a technician to adjust the position of the patient 32 resting on the table 30. The patient 32 can be repositioned whether the table 30 is inside or outside of the examination region 24 and bore 26. In general, the patient 32 is repositioned by inflating or deflating any one or more of the cells 78, 80. It is often desirable, and sometimes necessary, to slightly adjust the position of patient 32 to achieve better contact with and more accurate positioning relative to the transducer 44 during a MR guided HIFU therapeutic procedure. It may also be beneficial to slightly adjust the position of the patient within a MR imaging machine or other type of diagnostic machine as well. The adjustable support device 52 and the patient positioning system 50 disclosed and described herein may be well-suited for these types of machines and procedures, though they are particularly well-suited for MR guided HIFU procedures.

With reference to FIG. 2, all of the cells 78, 80 of the inflatable bed 76 in this example are completely inflated. The platform 82, and thus the patient 32, would achieve a specific position and orientation within the bore 26 with all of the cells inflated. The patient 32 can be repositioned relative to the transducer 44 as well as the table 30 by changing the level of inflation in any one or more of the cells 78, 80. The technician can manipulate the controls 60 and utilize the display 58 of the control apparatus 54 in order to do so. By inflating or deflating the cells 78, 80 of the inflatable bed 76 independently, the patient 32 can experience any number of positional adjustments. Also, the patient position can initially be a home position determined with all or some of the cells deflated, inflated, or partially inflated. Position adjustment can then be performed from the home condition instead of the fully inflated configuration of FIG. 2.

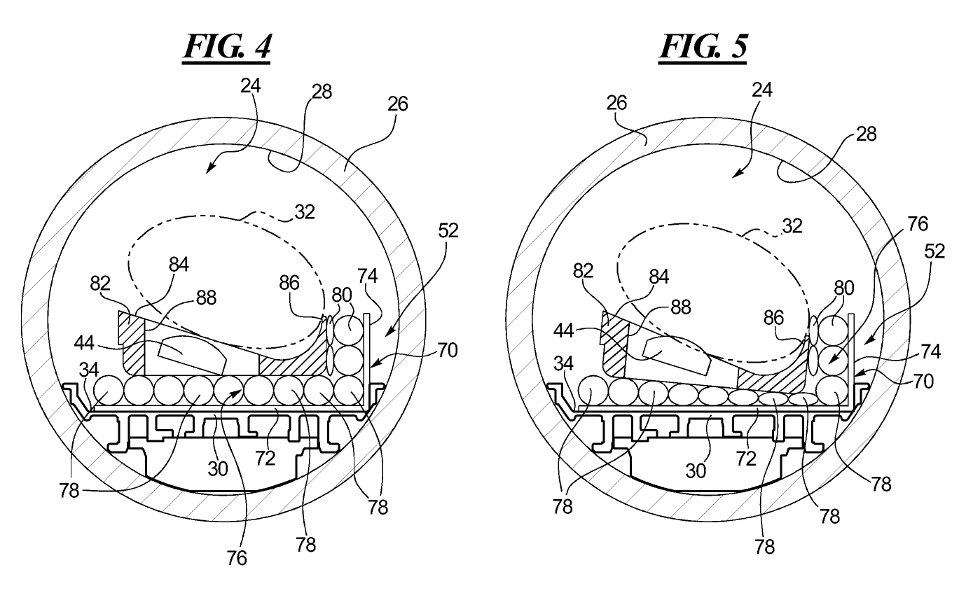

The cells 78, 80 can be adjusted to achieve virtually any combination of individual cell inflation/deflation condition. For example, FIG. 4 shows that one of the columns of inflatable cells 80 can be deflated to alter the side-to-side position of the patient 32 relative to the table 30 and the HIFU transducer 44. FIG. 5 shows that the row of cells 78 can be progressively more deflated from one side to the other of the support device 52. In doing so, the platform 82, and thus the patient 32 will experience rotational adjustment relative to the lengthwise axis of the table 30 and hence the central axis C. These positional adjustments can be made to bring a portion of the body of the patient 32 into closer contact with the HIFU transducer 44 or to redirect the transducer relative to a portion of the patient's body.

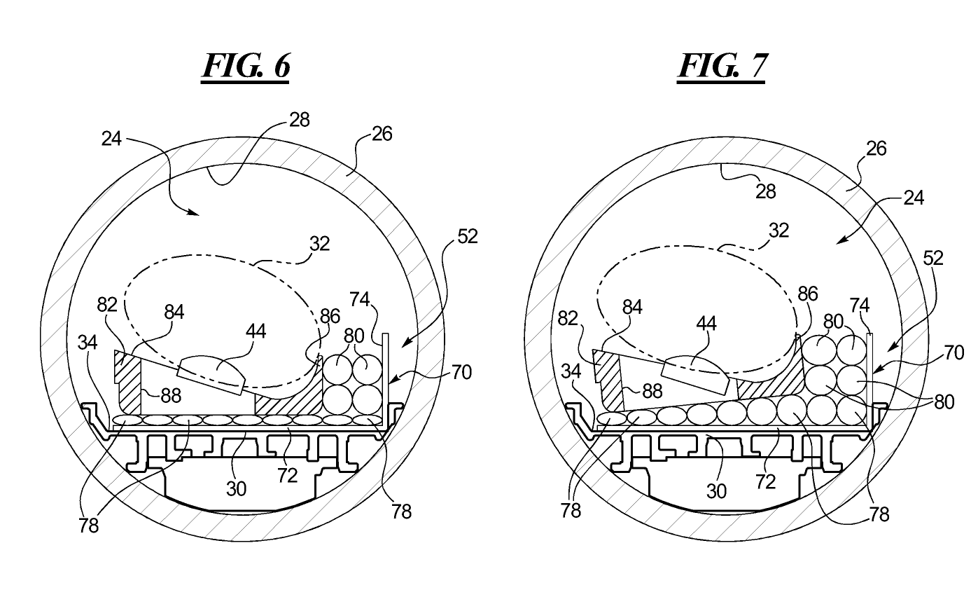

FIG. 6 shows another different example of position adjustment of the patient 32. In this example, both of the columns of cells 80 are fully inflated and the single row of cells 78 are uniformly deflated beneath the patient 32. The inflatable bed 76 can be manipulated in this manner to simply lower the patient position relative to the table 30 as needed. FIG. 7 shows that the row of cells 78 can also be progressively deflated from the other side to the one side of the support device 52, in a direction opposite to that represented in FIG. 5, to reorient the patient 32 about the lengthwise axis. Again, these positional adjustments to the patient 32 can be performed easily and efficiently in order to alter the position of patient relative to the HIFU transducer 44 as needed.

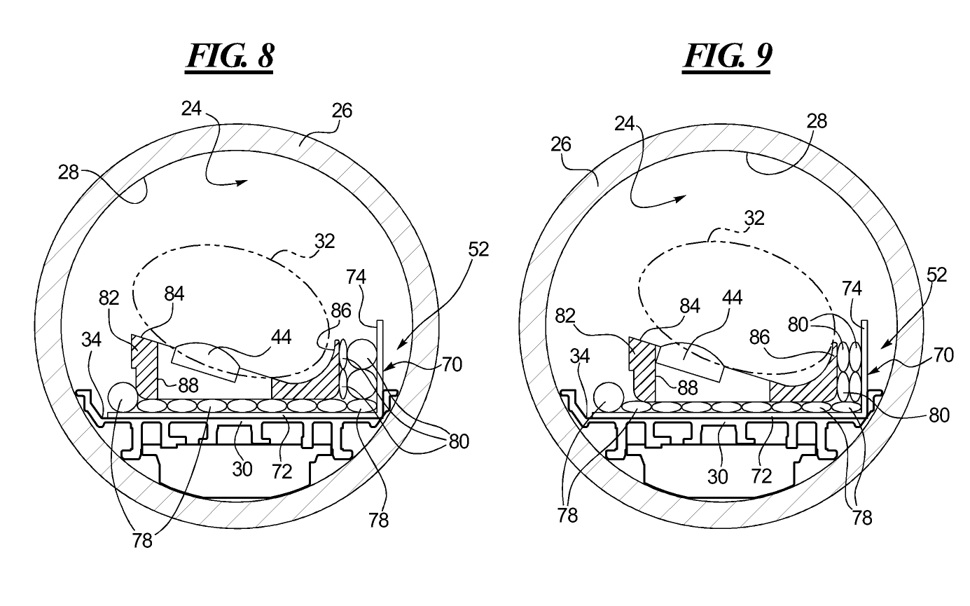

FIGS. 8 and 9 illustrate still further examples of different alternate positions of the patient 32 that can be achieved utilizing the support device 52 in this example. FIG. 8 simply shows that one of the columns of cells 80 and the row of cells 78 can be uniformly deflated, other than one of the cells, which is furthest from the columns of cells and not positioned beneath the platform 82. FIG. 9 shows the inflatable bed 76 in essentially the same condition as that depicted in FIG. 8 except that both of the columns of cells 80 are deflated.

As will be evident to those having ordinary skill in the art upon reading this disclosure, a vast array of alternate patient positions can be achieved utilizing the inflatable patient positioning system 50 and support device 52 disclosed herein. Repositioning of the patient can also be performed efficiently because the patient need not get off of the table 30 while the support device 52 is being adjusted. Also many patients being treated during a HIFU procedure may be sedated and hence cannot on their own reposition their body on the table. Repositioning of the patient can also be performed at any time when the patient is lying on the table 30, whether the table is inside or outside of the examination region 24 of the MR machine 20.

A number of other examples of patient positioning systems and support devices are possible within the spirit and scope of the present invention. Several examples are now described to illustrate. However, examples disclosed herein are not intended to limit the invention in any way to only those examples. Other alternate examples are possible as well. In the foregoing example, the fluid fillable cells 78 and 80 are depicted as being circular. It is certainly possible that the inflatable cells take on other shapes when fully inflated and viewed in cross-section. In addition, the cells 78 and 80 described above can each be a single elongated chamber extending the entire length of inflatable bed 76 and/or support device 52. Alternatively, the inflatable cells can be segmented lengthwise as well as widthwise relative to the support device 52 as desired. In yet another example, the row of cells 78 beneath the patient 32 or platform 82 can include only two or a few laterally spaced apart and/or lengthwise spaced apart cells and yet function adequately.

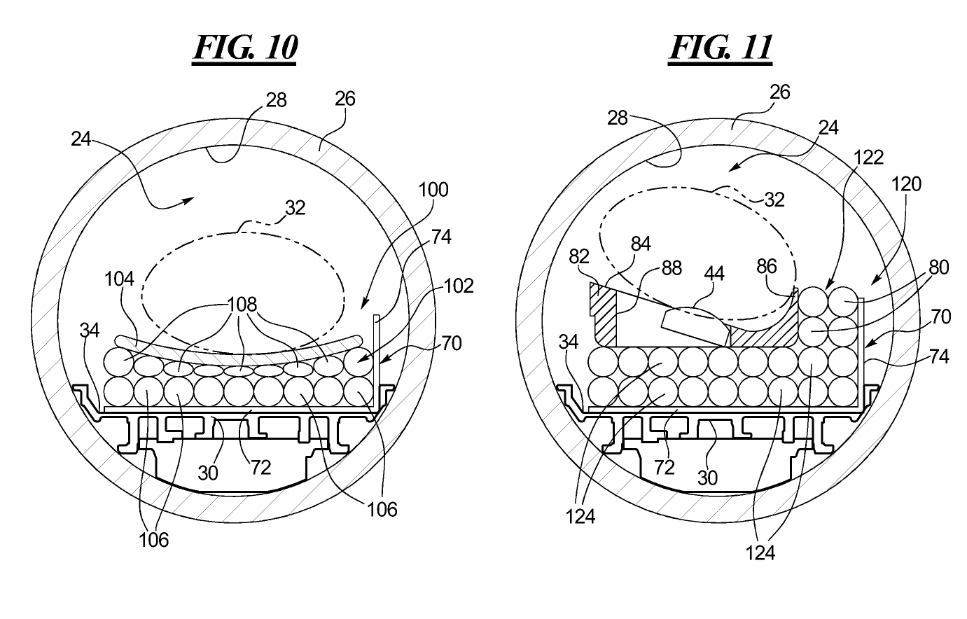

In each of the following examples, like reference numbers refer to like parts between the previously described example and following examples. FIG. 10 shows one alternate example of a support device 100 constructed in accordance with the teachings of the present invention. The support device 100 has an inflatable bed 102 carried on or mounted to the carrier 70. The earlier described wedge-shape platform 82 is replaced in this example by a symmetrically shaped, slightly concave, curved platform 104 resting on the inflatable bed 102. In this example, the inflatable bed 102 includes two rows or layers of fluid fillable cells 106, 108 positioned between the platform 104 and the carrier 70. In order to accommodate the curved shape of the platform 104, the top layer of cells 108 can each be configured having a non-circular, inflated, cross-sectional shape and can be different in shape from one another or the cells could be sequentially inflated with different pressures to achieve the same concave effect. Thus, the inflatable bed 102 can be configured to define a concave curved top surface to match that of the curved platform 104 so as to provide evenly distributed support for the platform when fully inflated.

FIG. 11 shows another alternate example of a support device 120 constructed in accordance with the teachings of the present invention. The only difference between this example and the earlier described support device 52 is found in the inflatable bed. The support device 120 has an inflatable bed 122 with two layers or rows of cells 124 between the platform 82 and the carrier 70, thus allowing for even more positioning ranges in translation, curving, raising or lowering, and the like. The inflatable bed 122 in this example has the same two columns of cells 80 along one side.

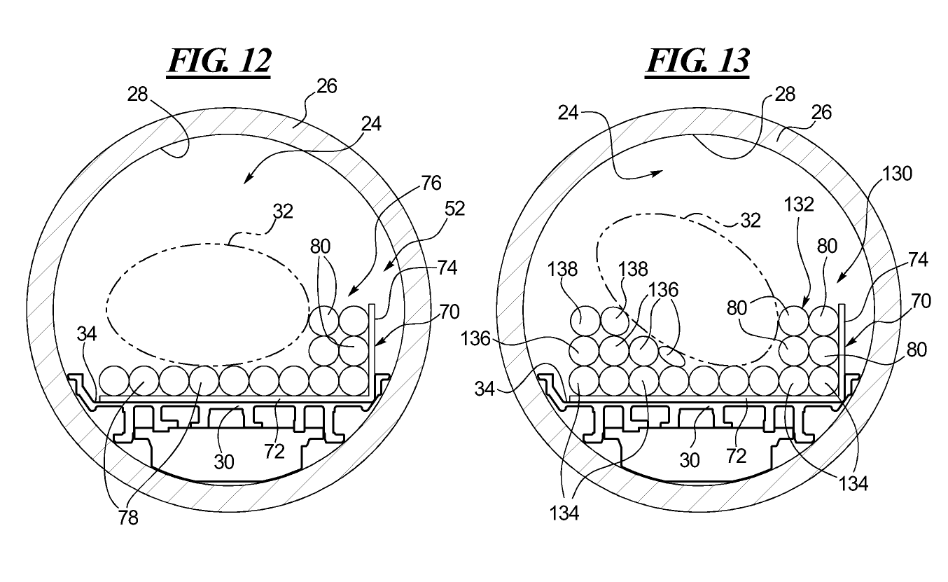

FIG. 12 shows that the disclosed support devices, such as the support device 52, may be provided or utilized without any intervening platform, such as the platform 82, between the patient 32 and the inflatable bed, such as the inflatable bed 76. In this example, the patient 32 lies directly on the row of cells 78 of the inflatable bed 76 and against one of the columns of cells 80. The embodiment of FIG. 12 is merely a representative example. It is possible that the patient 32 can lie against the inflatable cells of any one of the various other inflatable beds of the support devices disclosed and described herein, as well as other alternative examples not shown or described herein.

FIG. 13 similarly shows that the cells of the inflatable bed need not define a generally horizontal support surface, as does the bed in the prior example of FIG. 12. In this example, a support device 130 has an inflatable bed 132, which does not include any type of patient platform, but does create an angled top surface. The inflatable bed 132 in this example has a bottom row of consistently shaped, circular inflatable cells 134 lying against the carrier 70. The inflatable bed 132 has a partial row of cells 136 disposed on top of the bottom row and shifted to one side. A smaller number of cells 138 define a third row of cells on top of the partial row of cells 136. The inflatable bed 132 in this example also includes two columns of cells 80 disposed against the vertical panel 74 of the carrier as in the prior examples. The partial rows of cells 136, 138 are positioned adjacent the edge of the inflatable bed 132 opposite the columns of cells 80.

The combination of the bottom row of cells 134 and the two partial rows of cells 136, 138 define a side-to-side angled or wedge shaped top surface, similar to the angled top surface 84 of the platform 82 described above. However, in this example, it is the inflatable bed 132 itself that defines the angled top surface. The angular orientation of the patient 32 lying on the inflatable bed 132 in this example can be adjusted and controlled by inflating and deflating various ones of the cells in the layers 134, 136, and 138. As in some of the previous examples, any one or more of the cells of the inflatable bed 132 in this example can include a non-circular cross-section shape to aid in further defining a desired top surface contour and orientation when all of the cells are fully inflated. In one alternate embodiment, it is possible that the makeup of cells shown in FIG. 13 could be composed of a number of complete layers of identical cells, such as, for instance, three layers of cells. By providing individual inflation control and regulation to each of the individual cells, the patient position as shown in FIG. 13 could be achieved by selective inflation/deflation of the cells and not require a specific arrangement of cells as described above.

In all of the previous examples described herein, the inflatable beds can include a fewer number of cells strategically placed on a carrier and/or beneath a platform or the patient to adjust the position of the patient relative to the table 30 and/or the HIFU transducer 44. The fluid fillable cells can be independent and discrete from one another and not connected to one another. However, in order to affect accurate and repeatable positioning of the patient, the cells can be connected directly to the table 30 or to the carrier 70 at strategic points. This can ensure that the inflatable cells remain fixed in position at all times during use.

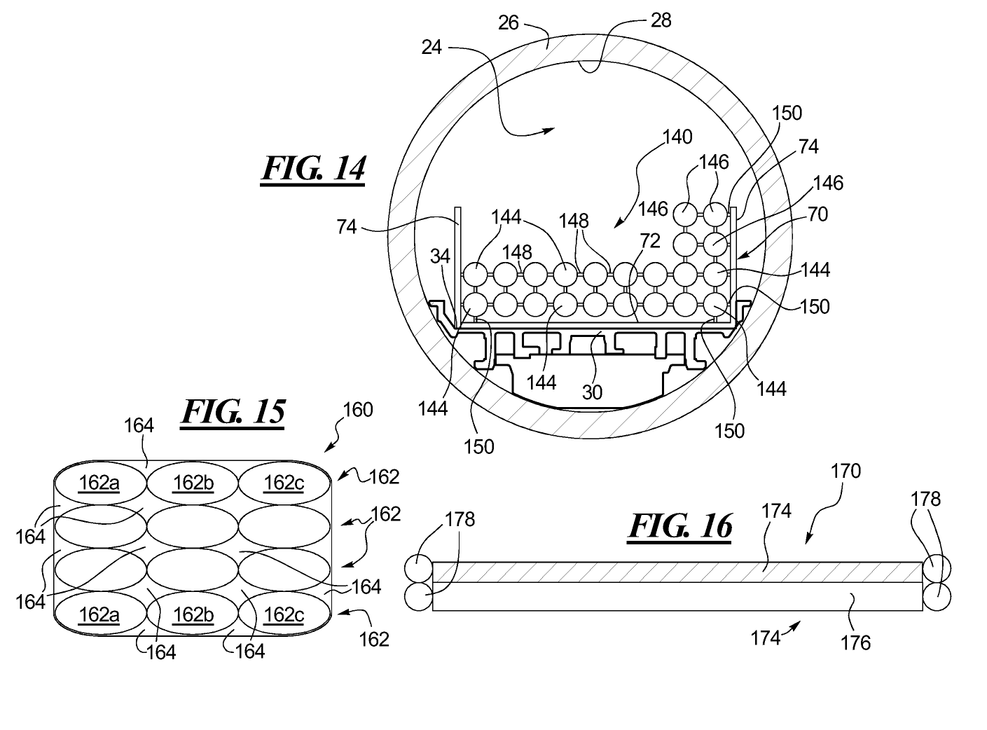

In the examples disclosed herein, the inflatable cells are directly adjacent other like inflatable cells. Adjacent cells can be connected directly to one another by an exoskeleton structure or the like in order to also aid in maintaining precise positioning of the inflatable cells during use. The columns of cells that provide lateral positioning of the patient can also be connected to one another and to some fixed surface, such as the one or more vertical panels 74 on the carrier 70 described herein. A mirror image column of cells (and mirror image vertical panel 74 as in FIG. 14) may be provided on the opposite side of the patient in any one or more of the examples disclosed and described herein to better provide for repositioning of a patient in either side-to-side direction, if desired.

Connecting the cells can be achieved by using RF welding techniques to permanently fuse the cells to each other in a controlled and selected width of material along the cell length. The cells can be constructed of any number of elastomeric covered fabrics. In one example, nylon woven fabric can be impregnated on one or both sides with thermoplastic polyurethane. Using metal fixtures or bars to clamp two pieces of coated fabric together, RF energy can be introduced to thermally bond two pieces of coated fabric together at a first selected joint along the length of a cell for example. Additional lengthwise bonds can be made to develop the required number of cells. A second RF weld can be made with the fabric to close the cell into a tube similarly along the length, this time at the top or bottom of the cell. Air inflation valves and fittings can similarly be welded onto the fabric and finally the ends can be RF welded to create each row of the sealed cells. Subsequent rows of the cells can be adhered to each other with an adhesive to create multiple connected rows for a complete, adjustable, patient support device. It should be noted that this construction is not limited to a single type of material or material coating, as fabrics of polyester, Kevlar and fiberglass or other materials can be laminated with any number of thermoplastic elastomers such as Teflon (PTFE), PVC or urethanes.

FIG. 14 shows a simplified cross-section or end view of another example of a support device 140 that has an inflatable bed 142 mounted to a carrier 70. The carrier 70 is again resting or connected to the top side 34 of the table 30. The inflatable bed 142 has two rows of cells 144 lying against the horizontal panel 72 of the carrier 70. Inflatable bed 142 also has two columns of inflatable cells 146 positioned adjacent the vertical panel 74. The inflatable bed 142 has a schematically illustrated interconnecting exoskeleton in this example. Adjacent cells 144, 146 are joined to one another by webs 148. Likewise, cells 146 that are adjacent the carrier 70 are joined or otherwise tethered to the carrier by webs 150. These webs 148 and 150 can vary considerably in number, location, configuration, and construction and yet function as intended. In one example, the webs 148, 150 can simply be welded seams of the inflatable bed material that define the parameters and edges of each individual cell 144, 146 and of the inflatable bed 142 itself. In one example, two layers of a suitable fabric or sheet material can be joined to one another along their perimeter edges and lengthwise along the material to define and form the various cells 144, 146 using the RF welding technique as previously described. The webs 148, 150 can secure the inflatable bed 142 at strategic points, as desired, to the table, carrier or the like.

In each of the above-described examples, the support devices include an inflatable bed with fluid fillable cells, bladders, or chambers that may extend essentially the entire length of the bed or the support device. Alternatively, the cells can be smaller and used in conjunction with existing pads on an MR table to be optimized for the various treatment needed for each individual patient. One can imagine, as noted above, that it is possible to provide an inflatable bed with multiple cell segments aligned in series over the length of the inflatable bed. FIG. 15 shows a generic representation of one such inflatable bed 160. The inflatable bed 160 has four widthwise rows of cells 162. Each row of cells 162 is further divided into three separate and discrete sub-cells or segments a, b, and c. Each of the individual cells 162a-c is separated by welded regions or seams 164 between the cells. As will be evident to those having ordinary skill in the art, it is possible that the support devices disclosed and described herein can include inflatable beds with cells of a variety of shapes, configurations, and arrangements as well as being joined with RF welding or adhesive or other means in a variety of different ways and configurations. The cells can be spherical, oval, elliptical, rectangular, square in cross-section, either lengthwise and/or width-wise. It is also possible that the support devices include only separate and discrete independent inflatable cells that are not connected in any way to one another or these separate cells can be contained within one outer piece of fabric or other support structure to create a similar inflation element or bed.

Utilizing the inflatable bed 160 of FIG. 15, one can individually inflate or deflate a width-wise row of cells 162 to alter the width-wise angle of a patient about the z- or lengthwise axis C. One can also individually inflate or deflate one or more of the lengthwise row of cells 162a-c to alter the lengthwise angle of the patient about the lateral or y-axis across the table. Thus, the disclosed support devices can be configured to adjust the position of a patient from one end to the other as well as from side to side.

The support devices disclosed and described herein can also include lengthwise or head-to-toe adjustment capability. FIG. 16 shows a generic example of a support device 170 having an inflatable bed 172, minus any type of carrier underlying the bed. A platform 174 rests on the top surface of the inflatable bed 172. The inflatable bed 172 has rows of lengthwise oriented cells 176 underlying the platform, though only one such cell is shown. The inflatable bed also has a column of widthwise oriented cells 178 positioned at each lengthwise end of the inflatable bed 172. The cells 178 can be inflated and deflated as necessary to move a patient in a lengthwise direction along the central axis C of the MR machine and relative to the table 30 and HIFU transducer 44 (each not shown). The cells 178 can also be connected or coupled to a fixed portion of the support device, such as end wall of a carrier (not shown) to help impart motion to the inflatable bed and thus the patient when these cells are inflated or deflated. A separate, independent set of cells, bladders, or pistons using the same fluid as the inflatable bed could instead be utilized. The motion induced by the support device shown in FIG. 16 can mimic that of a linear actuator and yet still be compatible for use in the MR machine environment.

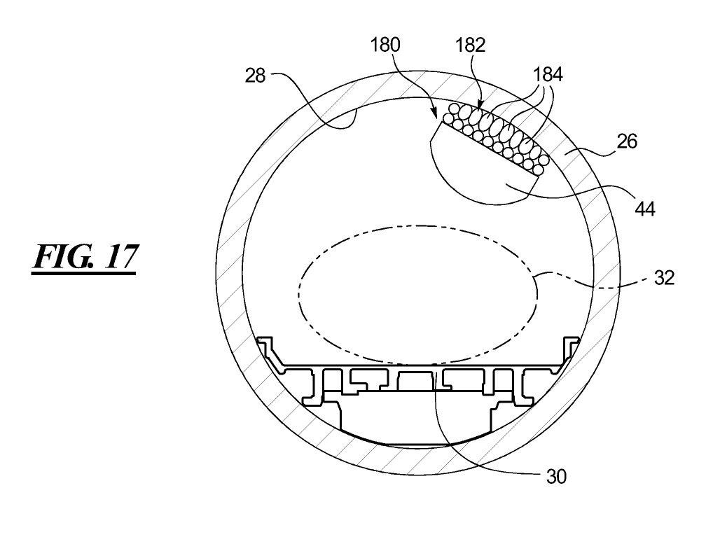

FIG. 17 shows one alternative example of a support device, system, and method according to the teachings of the present invention. In this example, the patient rests on a stationary table 30. Instead, the HIFU transducer 44 is mounted to a support device 180. The support device 180 has an inflatable element 182, which can include one or more fluid fillable cells 184, in virtually any of the configurations described above. The support device 180 is mounted to the interior wall 28 of the bore 26. Once the patient is moved into the bore 26, the inflatable element 182 can be controlled to adjust the position of the HIFU transducer 44, while the patient lies still. The cells 184 can be inflated and/or deflated as needed to position and press the HIFU transducer against the patient. This arrangement can achieve essentially the same result as the previously describe support device examples.

A selectively inflatable support device on a table or transport of a medical imaging machine, such as a MR machine, allows for quick and easy repositioning of the MR patient. Repositioning can be done directly within the bore of the MR machine. The disclose methods, patient positioning systems, and support devices can be used for any MR procedure but are particularly useful during MR guided HIFU therapy treatments. Such treatments often require fine tuning the position of the patient tissue to achieve optimal results or require precisely re-positioning a patient identically to a previously conducted diagnostic or therapeutic procedure on the same patient. With MR guide HIFU, the tissue of the patient to be treated must be placed, positioned, and oriented accurately relative to the HIFU transducer, whether in a supine or prone position. In such procedures, the patient must often be twisted slightly to provide better access to organs or internal tissue. Thus, wedge-shaped or other custom shaped platforms optimized for the individual treatment procedure may be provided as an optional part of the disclosed support devices and systems.

Patients vary greatly in size and weight. Thus, adjustability in angle, elevation, orientation or tilt, and linear position should or must be readily available to undergo time efficient and highly effective treatments and procedures. The disclosed support devices include inflatable cells or bladders to adjust patient position and height within the bore of the MR machine. The cells or bladders can be constructed of a flexible but tough fabric or sheet, such as polyurethane coated nylon or other fabric materials and thermoplastic or thermoset coatings. The cells or bladders can be fabricated by laminating two layers of fabric or sheet material using standard adhesive techniques, RF welding, or the like. A robust seal should be achieved so that the cells or bladders are independently stable when inflated. The cells or bladders can be of virtually any size and shape as well. The separate rows, columns, and the like of the cells or bladders can be controlled individually and independent of the other cells or bladders. The patient can then be lifted, lowered, tilted or reoriented, moved side-to-side, and/or moved lengthwise relative to the table or to a fixed set of HIFU transducers.

A composite or plastic exoskeleton frame can be configured to support the cells or bladders at strategic points around at least the perimeter of the support device. The support device or table can also allow for predetermined positioning and support for the fluid lines. The remaining components of the control apparatus, such as the pump or compressor, can be located outside of the MR suite in a separate control room. The fluid lines could then be routed from the pump or compressor and control valves to the MR suite through a wall feed-through, typically known as a wave guide. One or more of the pumps or compressors could be utilized. Air is the preferred fluid for inflating the cells or bladders. However, other gaseous or liquid fluids could be used as well.

A computer or processor can be coupled to the user interface, the one or more pumps or compressors, the controller, and/or the control valves. The display or graphical interface can allow a MR technician to control and enable easy adjustment of patient position. The position of the patient tissue can be readily verified by a quick MR scan.

The ultrasound transducer or transducers represented herein as the HIFU transducer 44 can be any now known or later developed ultrasound device. For example, the ultrasound transducer 44 can be a single element stationary type, a mechanically steered type, or a multi-element array using electronic steering. The transducer 44 can convert between acoustic and electrical energies. The ultrasound transducer may include, but is not to be limited to, transmit and receive beamformers, which relatively delay and apodize signals for different elements of the transducer 44. B-mode, Doppler, or other detection may or may not be utilized and performed on the beamformed signals. A scan converter, memory, three-dimensional imaging processor, and/or other components may be provided, along with high powered transformers to deliver adequate power to the ultrasound transducer for HIFU treatments

The transducer 44 can be a one-, two-, or multi-dimensional array of piezoelectric or capacitive membrane elements. The transducer 44 could be of a handheld type or, more likely, a machine held type for positioning against and outside of the patient.

In another example, the control apparatus can be programmed to automatically micro-adjust the patient position or the HIFU transducer position to account for minor and periodic patient movement detected by the system during a procedure. Though not shown herein, sensors in the table, the support device, the inflatable element, or elsewhere in the machine could be positioned and configured to detect motion created by the patient's breathing or involuntary movements. The sensors could signal the control apparatus to automatically, continuously, and or immediately make minor adjustments to pressure within individual cells to account for and counter such movements. This can be done to achieve even better, more consistent results and to reduce patient treatment time by potentially eliminating the need for delay-causing motion compensation during a procedure.

Various improvements described herein may be used together or separately. Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention.

I (we) claim:

1. A system for adjusting a position of a patient and a high intensity focused ultrasound (HIFU) transducer relative to one another in a medical imaging machine having a table, the system comprising:
an adjustable support device including an inflatable element; and
a control apparatus coupled to the adjustable support device and operable to inflate and deflate the inflatable element to alter the position of the patient supported by the table and the HIFU transducer relative to one another.

2. The system of claim 1 wherein the inflatable element includes one or more fluid fillable cells arranged to raise or lower the patient relative to the table.

3. The system of claim 2 wherein the one or more fluid fillable cells are arranged to adjust the orientation of the patient about a lengthwise axis of the table.

4. The system of claim 1 wherein the inflatable element includes at least one fluid fillable cell arranged to move a patient laterally relative to the table.

5. The system of claim 1 wherein the inflatable element includes at least one fluid fillable cell arranged to move a patient lengthwise along an axis of the table.

6. The system of claim 1 wherein the adjustable support device further comprises a carrier positioned beneath the inflatable element, and wherein portions of the inflatable element are secured to the carrier.

7. The system of claim 1 wherein the HIFU transducer is coupled to the adjustable support device and the adjustable support device is mounted to a surface within a bore of the medical imaging machine.

8. The system of claim 1 wherein the control apparatus comprises:
a pump or compressor;
one or more valves; and
one or more lines each in fluid communication with part of the inflatable element, the pump or compressor, and a corresponding valve of the one or more valves, the pump or compressor and valves operable to inflate and deflate the inflatable element of the adjustable support device.

9. The system of claim 1 wherein the inflatable element has one or more vertically adjacent layers of cells and/or has one or more horizontally adjacent layers of cells.

10. The system of claim 1 wherein the adjustable support device has a length and the inflatable element has one or more fluid fillable cells each having an interior fluid space extending substantially the length of the adjustable support device.

11. The system of claim 1 wherein the medical imaging machine is a magnetic resonance machine.

12. A support device for a medical imaging machine having a bore and a table movable into and out of the bore with a top side above which the patient is positioned, the support device comprising:
an inflatable element positioned above the top side of the table and having a plurality of fluid fillable cells that are selectively inflatable and deflatable to alter a position of a patent relative to the top side of the table.

13. The support device of claim 12 further comprising:
a patient platform carried on top of the plurality of fluid fillable cells.

14. The support device of claim 13 wherein the patient platform is a wedge shaped structure having a top surface oriented at a side-to-side angle relative to a horizontal reference and the top side of the table.

15. The support device of claim 12 wherein the inflatable element includes at least one fluid fillable cell positioned along a side of the table arranged to laterally move or position a patient side to side when inflated or deflated.

16. The support device of claim 12 wherein the inflatable element includes at least one fluid fillable cell positioned along a lengthwise end of the table arranged to move or position a patient in a lengthwise direction when inflated or deflated.

17. The support device of claim 12 wherein a substantial portion of the plurality of fluid fillable cells are positioned and arranged to raise and lower a patient relative to the top side of the table and/or to alter an angle of a patient about a lengthwise axis and/or about a width-wise axis of the support device when inflated and deflated.

18. The support device of claim 12 further comprising a carrier mounted to the table, the inflatable element connected to the carrier.

19. The support device of claim 12 wherein a portion of the plurality of fluid fillable cells are secured to the table or to a carrier on the table to maintain a position of the cells relative to the table.

20. A method of positioning a patient on a table movable into and out of a bore of a medical imaging machine, the method comprising:
mounting an adjustable support device above a top side of the table, the adjustable support device having one or more fluid fillable cells;
positioning a patient on the adjustable support device; and
operating a control apparatus to selectively inflate, deflate, or both, any one or more of the one or more of the fluid fillable cells to adjust the position of the patient relative to the top side of the table.

21. A method of positioning a HIFU transducer and a patient relative to one another within a bore of a magnetic resonance machine, the method comprising:
positioning a patient on a table;
moving the table into the bore; and
operating a control apparatus to selectively inflate, deflate, or both, any one or more of a plurality of fluid fillable cells to adjust the position of the patient and HIFU transducer relative to one another.

22. The method of claim 21 wherein the step of operating the control apparatus further includes moving the patient relative to the HIFU transducer.

23. The method of claim 21 wherein the step of operating the control apparatus further includes moving the HIFU transducer relative to the patient.
